(19) United States
(12) Patent Application Publication
(10) Pub. No.: US 2003/0083250 A1
(43) Pub. Date: May 1, 2003

(54) Anti-tumor chemotherapy by administration of cyclophosphamide and erythropoeitin

(76) Inventors: Francis Farrell, Doylestown, PA (US); Oliver Thews, Mainz (DE); Peter Vaupel, Mainz (DE)

Correspondence Address: JANET E. REED ,ESQ, WOODCOCK WASHBURN LLP, ONE LIBERTY PLACE 46TH FLR., PHILADELPHIA, PA, 19103, US

(21) Appl. No.: 10/001,666

(22) Filed: Oct. 25, 2001

(51) Int. Cl. A61K 38/22; A61K 31/66

(52) U.S. Cl. 514/12; 514/105

(57) ABSTRACT

The present invention provides, in one embodiment, methods to treat, prevent the progression of, or facilitate elimination of a malignancy by increasing the supply of red blood cells using erythropoietin in conjunction with administration of an anti-tumor agent.

### FIELD OF THE INVENTION

The present invention provides, in one embodiment, methods to treat, prevent the progression of, or facilitate elimination of a malignancy by increasing the supply of red blood cells using erythropoietin in conjunction with administration of cyclophosphamide. In one embodiment, methods are provided that by administration of erythropoietin that results in complete amelioration of anemia prior to administration of cyclophosphamide. In another embodiment, erythropoietin is administered concurrently with cyclophosphamide. In yet another embodiment, methods are provided where the red blood supply is raised to super-physiological (is super-physiological right? Don't we mean “raised above each patient or subject's baseline”) levels by administration of erythropoietin prior to or concurrently with administration of cyclophosphamide to facilitate eradication of a tumor?

### BACKGROUND OF THE INVENTION

PROCRIT® is the brand name for the Epoetin alfa. In 1990, PROCRIT received market clearance by the FDA for the treatment of anemia in HIV-infected patients on zidovudine (ZDV) therapy (≦4200 mg/week) with endogenous serum erythropoietin levels ≦500 MU/mL. It is also approved for the treatment of anemia in patients with non-myeloid malignancies receiving chemotherapy, in patients with chronic renal failure (pre-dialysis), and for use in elective noncardiac nonvascular surgery to reduce the need for allogeneic blood transfusion during high volume blood loss procedures. In clinical trials to date, Epoetin alfa has been evaluated in normal subjects as well as in subjects with various anemic conditions. Epoetin alfa induces a brisk haematological response in normal human volunteers, provided that adequate supplies of iron are available to support increased hemoglobin synthesis. A majority of trials have investigated the safety and effectiveness of Epoetin alfa in the treatment of chronic renal failure and of anemia in cancer. Other trials have evaluated Epoetin alfa for the treatment of anemia associated with rheumatoid arthritis, prematurity, AIDS, bone marrow transplantation, myelofibrosis, sickle cell anemia, as a facilitator of presurgical autologous blood donation, and as a perisurgical adjuvant.

Erythropoietin is currently used to treat anemic subjects who are amenia as a result of insufficient levels of Erythropoietin or who demonstrate a blunted response to Erythropoietin. Erythropoietin is not currently used to treat hemolytic anemia or most form of anemia that results from enhanced rate of clearance of the red blood cells, except for sickle cell anemia and thalycemia.

Epoetin alfa is approved for sale in many countries for the treatment of anemia in chronic renal failure (dialysis and predialysis), anemia in zidovudine treated HIV positive patients (US), anemia in cancer patients receiving platinum-based chemotherapy, as a facilitator of autologous blood pre-donation, and as a perisurgical adjuvant to reduce the likelihood of requiring allogeneic blood transfusions in patients undergoing orthopedic surgery.

Early clinical trials with Epoetin in cancer involved anemic patients with a variety of malignant diseases and have provided information on the effect of Epoetin on the hemoglobin concentrations, hematocrit and subsequent red blood cell transfusion requirements of such patients. Encouraging results were obtained following the administration of Epoetin alfa to anemic patients with solid cancers (C. B. Miller, L. C. Platanias and S. R. Mills, J Nat. Cancer Inst. (1992) 84:98-103), lymphoproliferative disorders such as malignant lymphoma and hematopoietic stem cell disorders (M. Cazzola, L. Ponchio and Y. Beguin, Blood (1992) 79:29-37), and multiple myeloma (B. Barlogie and T. Beck, Stem Cells (1993) 11:88-94; J. T. Beck, K. Hayden, and L. Hutchins, Proc. Am. Soc. Clin. Oncol., (1992) 11:Abstract 1228; H. Ludwig, E. Fritz, H. Kotzmann and H. Gisslinger, New Engl. J Med. (1990) 322:1693-1699; H. Ludwig, E. Fritz and C. Leitgeb, Ann Oncol (1993) 4:161-167).

Cancer is frequently associated with significant anemia, and has traditionally been treated by blood transfusion. Anemia may result from the disease itself, the effect of concomitantly administered chemotherapeutic agents, or a combination of both. The condition often takes on the characteristics of the anemia of chronic disease (ACD). ACD is associated with erythroid hypoplasia of the bone marrow, a somewhat shortened circulating life of red cells and decreased bone marrow re-utilization of iron. Clinical experience has been collected over the past years to show that Epoetin alfa can correct anemia in cancer patients at doses several times higher than those shown to be effective in renal patients. About 50% to 60% of anemic cancer patients receiving chemotherapy responded with a hemoglobin rise of at least 2 g/dL to Epoetin alfa therapy given three times weekly at a dose of 150 IU/kg over a period of twelve weeks (R. I. Abels, K. M. Larholt, K. D. Krantz and E. C. Bryant, Proceedings of the Beijing Symposium, Alpha Medical Press, Dayton, Ohio, (1991) 121-141). In a subsequent open-label dose titration study, doses up to 300 IU/kg, were sometimes required, demonstrating the relative resistance to the effect of erythropoietin in these patients. If erythropoietin levels are measured, they are found to be within the normal range, but inappropriately low for the degree of anemia: there is a blunted erythropoietin response.

In a series of controlled clinical trials, anemic cancer patients receiving cyclic platinum or non-platinum containing chemotherapy were treated with placebo or Epoetin alfa (150 IU/kg t.i.w.). In these trials, Epoetin alfa was shown to increase hematocrit and decrease transfusion requirements after the first month of therapy (R. I. Abels, K. M. Larholt, K. D. Krantz and E. C. Bryant, Proceedings of the Beijing Symposium. Alpha Medical Press, Dayton, Ohio, (1991) 121-141). In 1994, the Product Licence for Epoetin alfa was extended in several countries to include “The treatment of anemia in adult cancer patients receiving platinum containing chemotherapy regimens”. However, more data needed to be collected to establish the efficacy of Epoetin alfa in cancer patients treated with non-platinum and platinum containing chemotherapy.

For patients receiving platinum-based chemotherapy, the Committee for Proprietary Medicinal Products (CPMP)-approved Summary of Product Characteristics specifies that epoetin alfa is indicated to treat anemic patients with an initial dose of 150 IU/kg s.c. t.i.w. (Subcutaneously, three times weekly) and a target hemoglobin level of approximately 12 g/dL. After four weeks of therapy, the dose may be adjusted up to 300 IU/kg t.i.w. based on hemoglobin and reticulocyte response. Hemoglobin status should be checked periodically, and iron status should be evaluated prior to and during treatment; iron supplementation should be administered if necessary. Therapy should continue until one month after the end of chemotherapy.

Despite apparent success in using erythropoietin in conjunction with an anti-tumor therapy in treating anemia, there is room for improvement in terms of effective dosing regimens, patient benefit, and cost-effective use of EPO. According to a recent review of clinical trials involving EPO response rates in patients receiving platinum containing chemotherapeutics, the percentage of patients who responded to EPO ranged from 36 to 82% for all trials considered, and from 50 to 79% for trials with similar response criteria. This wide variety of response rate was partly due to clinical trial design, but was also attributed to lack of a strong predictor of EPO response in this patient population (T. Fjornes, J Oncol Pharm Practice (1999) 5(1):22-31). Clearly the blunted erythropoietin response in subjects with malignancy requires more study for effective dosing regimens.

Decreasing the onset or severity of anemia is a desirable effect of EPO dosing regimens, resulting in increased quality of life and reduced fatigue. However the ultimate goal of any chemotherapeutic regimen is to prevent progression of the malignancy, reduce or eliminate the primary tumor, and preserve the life of the patient. It has been proposed that, in addition to affecting the well being of the patient, EPO may act by reducing hypoxia (low oxygen), particularly tumor hypoxia, which is higher than in normal tissue. Most solid tumors exhibit physiological conditions different than normal tissue, including regions of hypoxia, low pH, and low glucose levels. This is thought to contribute to multi-drug resistance, including to the drugs etoposide, doxorubicin, camptothecin, and vincristine (T. Akihiro, T. Takashi, Anti-Cancer Drug Des. (1999) 14(2):169-177). In a recent report, SCID (severe combined immunodeficient) mice containing human ovarian cancer xenographs were examined for response to cisplatin versus cisplatin+EPO. For animals engrafted with small tumors, a significant improvement in tumor regression was seen in the group of mice receiving cisplatin+EPO compared with cisplatin alone, thus supporting the implication of oxygen sensitization of the tumor (D. F. Silver, M. S. Piver, Gynecol. Oncol. (1999) 73(2):280-284). However, this hypothesis has not been confirmed in human subjects, due to the complexity of the clinical trial conditions and the much higher costs associated with longer term trials.

International Patent Application WO9810650 describes a method of treating endothelial injury. The prior use of EPO with a chemotherapeutic agent is described for enhancing the chemotherapeutic efficacy with prior administration of an endothelial inhibiting amount of EPO.

International Patent Application WO9952543 describes pharmaceutical compounds comprising erythropoietin for the treatment of cancer. The concurrent use of EPO with a chemotherapeutic agent is not described. This publication describes a small, uncontrolled study of five (5) patients who received recombinant human erythropoietin for 14 to 85 weeks beyond an initial 12-week course of EPO treatment. The initial EPO treatment was conducted to evaluate safety and tolerance of EPO in multiple myeloma patients and is described by Mittleman et al., Acta Haematol. (1997) 98:204-210. The authors then conducted a series of mouse model experiments, where mice bearing a “progressive myeloma” were treated with various dosing regimens of recombinant human EPO. This reference does not teach dosing regimens suitable for treatment of human malignancies using EPO. Further it is not predictable that the results seen in a mouse myeloma model would translate into other tumor types, or would indicate success in humans. For example, in experiments conducted in SCID mice bearing human ovarian cancer, mice treated with EPO alone (a control group) showed no changes in tumor growth as a result of EPO administration (Silver et al. (1999)). These results described for the small, uncontrolled human experiments are not instructive in such that they lack sufficient control to allow for interpretation of the drug's (EPO) effects on the patient's health, specifically tumor response. This reference does not describe the use of EPO in a concurrent dosing regimen with another chemotherapeutic agent in order to enhance tumor response to the chemotherapeutic. The present invention provides the discovery that an EPO regimen in conjunction with a chemotherapeutic regimen provides greater tumor response compared to a chemotherapeutic regimen alone. This unmet need of improved tumor response could not be predicted from prior clinical studies that examined the effects of using an EPO regimen to prevent, improve, or ameliorate anemia or to improve quality of life. The present invention describes a method to treat patients who suffer from cancer and are either currently receiving or are scheduled to receive non-platinum and platinum containing chemotherapeutics concurrently with an improved EPO dosing regimen.

### SUMMARY OF THE INVENTION

The present invention provides a method to treat a subject in need thereof comprising the steps:

a) administration of a therapeutically effective amount of erythropoietin; and

b) administration of a therapeutically effective amount of cyclophosphamide,

wherein the subject has a solid tumor and where the increased hemoglobin level increases the therapeutic efficacy of the cyclophosphamide.

The method of the present invention is also drawn to include a method in which the erythropoietin is administered to the subject in the range of about 50 to 1000 IU/kg. The method the present invention is also drawn to include a method in which the erythropoietin is administered to a male subject to produce a hemoglobin level in the subject that is greater than about 15 g/dL. The method of the present invention is also drawn to include a method in which the erythropoietin is administered to a male subject to produce a hemoglobin level in the subject that is greater than about 16 g/dL. The method of the present invention is also drawn to include a method in which the erythropoietin is administered to a male subject to produce a hemoglobin level in the subject that is greater than about 17 g/dL. The method of the present invention is also drawn to include a method in which the erythropoietin is administered to a female subject to produce a hemoglobin level in the subject that is greater than about 13 g/dL. The method of the present invention is also drawn to include a method in which the erythropoietin is administered to a female subject to produce a hemoglobin level in the subject that is greater than about 14 g/dL. The method of the present invention is also drawn to include a method in which the erythropoietin is administered to a female subject to produce a hemoglobin level in the subject that is greater than about 15 g/dL.

### BRIEF DESCRIPTION OF THE DRAWINGS

FIG. 1: Time course of hemoglobin concentrations (cHb) in animals treated only with a single dose of carboplatin on Day−4 (♦), in animals additionally treated with rHuEPO (from Day−11 until Day+5, three times a week) (◯), and in non-anemic control animals (Δ). Day 0 is the day of tumor implantation. Each point represents data from at least 22 animals. Arrows indicate the times of rHuEPO application (EPO), anemia induction (AI) by carboplatin injection, as well as tumor implantation (TI) and cyclophosphamide treatment. (**) p<0.01.

FIG. 2: Tumor volume in animals treated only with a single dose of carboplatin for anemia induction 4 days before tumor implantation (♦), in animals additionally treated with rHuEPO (◯), and in non-anemic control animals (Δ). Each data point represents the mean tumor volume of at least 16 tumors.

FIG. 3: Tumor volume in animals with carboplatin-induced anemia (♦), in animals where the development of anemia was prevented by rHuEPO treatment (◯), and in non-anemic controls (Δ). All tumors were treated with a single dose of cyclophosphamide (60 mg/kg, i.p.) on Day 5. Each data point represents the mean tumor volume of at least 14 tumors. (*) p<0.05, (**) p<0.01. For the comparison of anemic animals with the group where anemia was prevented by rHuEPO treatment.

### DETAILED DESCRIPTION OF THE INVENTION

### Definitions

The erythropoietin is present in the compositions in therapeutically effective amounts. “Erythropoietin” shall include those polypeptides and proteins that have the biological activity of human erythropoietin, as well as erythropoietin analogs, erythropoietin isoforms, erythropoietin mimetics, erythropoietin fragments, hybrid erythropoietin proteins, fusion proteins oligomers and multimers of the above, homologues of the above, glycosylation pattern variants of the above, and muteins of the above, regardless of the biological activity of same, and further regardless of the method of synthesis or manufacture thereof including but not limited to, recombinant whether produced from cDNA or genomic DNA, synthetic, transgenic, and gene activated methods. Specific examples of erythropoietin include, Epoetin alfa (EPREX®, ERYPO®, PROCRIT®), Novel erythropoiesis stimulating protein (NESP) (a hyperglycosylated analog of recombinant human erythropoietin (Epoetin) described in European patent application EP640619), human erythropoietin analog—human serum albumin fusion proteins described in the international patent application WO9966054, erythropoietin mutants described in the international patent application WO9938890, erythropoietin omega, which may be produced from an Apa I restriction fragment of the human erythropoietin gene described in U.S. Pat. No. 5,688,679, altered glycosylated human erythropoietin described in the international patent application WO9911781, PEG conjugated erythropoietin analogs described in WO9805363 or U.S. Pat. No. 5,643,575.

Specific examples of cell lines modified for expression of endogenous human erythropoietin are described in international patent applications WO9905268 and WO9412650. Peptide mimetics of erythropoietin, abbreviated as “EMP” herein, are described in pending U.S. patent application Ser. No. 08/484135, filed on Jun. 7, 1995 by Zivin et al, the contents of which are herein incorporated by reference. The generally preferred form of EPO is purified, recombinant human EPO (rhEPO), distributed under the trademarks of EPREX®, ERYPO®, or PROCRIT®. Epoetin alfa is a sterile, clear, colourless, aqueous solution for injection, which is provided in prefilled, single-use or multi-dose quantities.

“Anemia” is a condition marked by decreases in hemoglobin (Hb) levels defined herein as ≦15.0 g/dL (9.30 mmol/l) for human male subjects and ≦13.0 g/dL (8.06 mmol/l) for human female subjects. Mildly anemic conditions are defined herein as Hb level ≦13.0 g/dL (8.06 mmol/l) for human males and ≦12.0 g/dL (7.44 mmol/l) for human females. Severely anemic conditions are defined herein for both human sexes as Hb ≦10.5 g/dL, with further medical intervention, typically in the form of blood transfusion, commonly administered at Hb <9 g/dL, although transfusions are not common at hemoglobin levels above 9.0 g/dL. It is assumed that worsening anemia is a likely consequence of further anti-tumor therapy and/or the underlying disease.

The term “concurrent” as used herein, means that a pharmaceutically effective amount of a EPO dosing regimen and an anti-tumor agent dosing regimen are administered during the same period of time such that the patient receives the benefit of both agents alone and achieves a synergistic effect of the combination of the two agents. Synergy refers to a combined pharmacological effect that exceeds the anticipated result based on the amount of administration of either single agent

The term “solid tumor” as used herein refers to the manifestation of a cancerous mass, as is well known in the art for example in Harrison's Principles of Internal Medicine, 14^{th} edition. For example, but not by way of limitation, solid tumors include cancers of the prostate, lung cancer, colorectal tissue, bladder, oropharyngeal/laryngeal tissue, kidney, breast, endometrium, ovary, cervix, stomach, pancrease, brain, and central nervous system.

The present invention provides a method to treat a subject in need thereof comprising the steps:

a) administration of a therapeutically effective amount of erythropoietin; and

b) administration of a therapeutically effective amount of cyclophosphamide

wherein the amount of erythropoietin administered is sufficient to produce at least a physiological hemoglobin level in the subject; and wherein the subject has a solid tumor.

In one embodiment, dosing regimens of EPO with cyclophosphamide are conducted preferably such that the subject is at the low range of normal physiological hemoglobin levels, about 15 g/dL for a male subject and about 13 g/dL for a female subject In another embodiment, erythropoietin is administered such that the subject produces a super-physiological amount of hemoglobin. The term “super-physiological” as used herein refers to a hemoglobin level of greater than 15 g/dL in a human male and greater than 13 g/dL for a human female.

Cyclophosphamide is administered within therapeutically effective amounts, which are well known in the art, and vary based on the type of malignancy, the state of health of the subject, and other conditions.

EPO is administered by any suitable means, as would be apparent to one skilled in the art. As used for administration of EPO, the phrase “therapeutically effective” is from about 1 to 1000 I.U./kg, preferably from about 50 to 1000 I.U./kg, more preferably from about 50 to 600 I.U./kg, and most preferably from 50 to 300 I.U./kg body weight, especially when erythropoietin is administered subcutaneously. The preferred methods of administration are intravenous (iv) and subcutaneous (sc), with subcutaneous being generally preferred. EPO is administered within the range of about 100 to 300 U/kg per dose, one to five times per week, or at any other dosing regimen that provides the desired therapeutic effect. A preferred initial dosing regimen is about 150 U/kg sc, three times per week, however it is readily apparent to those skilled in the art that any EPO dose or frequency of EPO administration that provides the therapeutic effect described herein is suitable for use in the present invention. For patients who show a blunted response to a dosing regimen of 150 I.U./kg, the preferred dosing regimen is about 300 I.U./kg sc, three times per week. EPO administration is delayed or withheld if the patient, male or female, exhibits a hemoglobin level in excess of about 18 g/dL for a human male and about 16 g/dL for a human female.

The invention can be better understood by way of the following examples. These examples are representative of the preferred embodiments, but are not to be construed as limiting the scope of the invention.

### EXAMPLE 1

### Materials and Methods

Animals:

Male Sprague-Dawley rats (Charles River Wiga, Sulzfeld, Germany; body weight 100 to 160 g) housed in our animal care facility were used in the study. Animals were allowed access to food and acidified water ad libitum before and throughout the investigation. All experiments had previously been approved by the regional animal ethics committee and were conducted in accordance with the German Law for Animal Protection and the UKCCCR Guidelines [United Kingdom Coordinating Committee on Cancer Research (UKCCCR) guidelines for the welfare of animals in experimental neoplasia (2 nd edit). Br. J. Cancer, (1998) 77:1-10].

Tumors:

Solid DS-sarcomas were induced by injecting DS-sarcoma cells (0.4 ml approximately 10⁴ cells/μl) subcutaneously into the hind food dorsum. Tumors grew as flat, spherical segments and replaced the subcutis and corium completely. Tumor volumes were determined by measuring the three orthogonal diameters of the tumor and using an ellipsoid approximation with the formula: V=d₁×d₂×d₃×π/6. From the volume growth curves the volume doubling time was calculated during exponential tumor growth.

Anemia Induction:

Prolonged anemia was induced by a single injection of carboplatin (Sigma-Aldrich, Steinheim, Germany) at a dose of 50 mg/kg body weight (dissolved in isotonic saline at a concentration of 20 mg/ml) into the tail vein four days before tumor implantation. The resulting anemia has been shown to last for at least ten days and to be sensitive to rHuEPO treatment [O. Thews, R. Koenig, D. K. Kelleher, J. Kutzner, P. Vaupel, “Enhanced Radiosensitivity in Experimental Tumours Following Erythropoietin Treatment of Chemotherapy-Induced Anaemia”, Br. J. Cancer, (1998) 78:752-756]. This carboplatin treatment regime does not affect the growth of DS sarcomas.

rHuEPO Treatment:

Recombinant human erythropoietin (epoetin alpha, ERYPO 4000®; Janssen-Cilag, Neuss, Germany) in isotonic NaCl solution was administered (1000 IU/kg) three times per week over sixteen days by s.c. injection starting eleven days before tumor implantation. Control animals received equivalent volumes of the solvent (0.5 ml/ kg).

Cyclophosphamide Treatment:

Tumors were treated on Day 5 after implantation when they had reached a volume of approximately 0.5 ml with a single dose of cyclophosphamide (Endoxan®, Asta, Frankfurt/Main, Germany; 60 mg/kg i.p.). This dose results in a growth delay of approximately eight to ten days, but not to be curative. Animals treated with equivalent volumes of the solvent (isotonic saline 6 ml/kg) served as controls.

Experimental Groups:

The experimental groups can be described as follows:

Group 1 (non-anemic): Animals received cyclophosphamide on Day 5 after tumor implantation.

Group 2 (anemic): Animals treated for four days before tumor implantation with carboplatin for anemia induction. Cyclophosphamide was administered on Day 5 after implantation.

Group 3 (anemic, rHuEPO-treated): Animals treated with rHuEPO three times per week from eleven days before tumor implantation and up until the day of cyclophosphamide application. Carboplatin (for anemia induction) was administered on Day −4 and cyclophosphamide was applied on Day 5.

Controls for all three experimental groups received equivalent volumes of the solvent instead of cyclophosphamide on Day 5 after tumor implantation.

Measurements:

Besides tumor growth, blood cell parameters were assessed using a multi-parameter, automated hematology analyzer (Ac-T₈; Beckman-Coulter, Krefeld, Germany) whereby erythrocyte, white blood cell and platelet counts together with the mean cell volume (MCV) were measured by an impedance technique and the hemoglobin concentration by a photometric method. In addition, the analyzer uses the measured values to calculate several other parameters (e.g., hematocrit, mean corpuscular hemoglobin content [MCH] and mean corpuscular hemoglobin concentration [MCHC]). All measurements were performed using a sample of venous blood (20 μl) taken from a tail vein.

Statistical Analysis:

Results are expressed as means±standard error of the mean (SEM). Differences between the groups were assessed by the two-tailed Wilcoxon test for unpaired samples. The significance level was set at α=5% for all comparisons. For characterizing the effect of chemotherapy on tumor growth, the growth delay induced by cyclophosphamide was calculated.

Results:

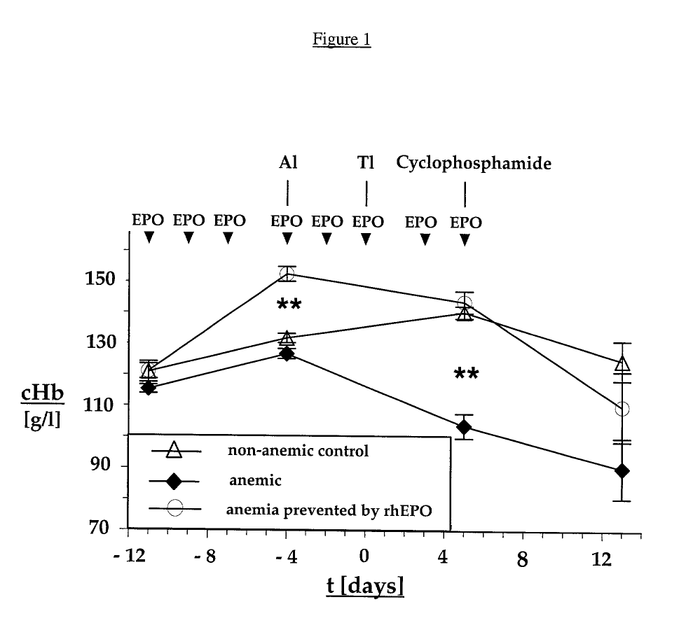

Carboplatin application four days before tumor implantation significantly reduced the hemoglobin concentration (cHb). Starting at a mean cHb of approximately 130 g/l at Day −4, a single dose of carboplatin of 50 mg/kg body weight i.v. resulted in pronounced anemia in rats with a mean cHb of about 100 g/l nine days after application. The hemoglobin content remained at lower levels for at least eight days (FIG. 1). Continuous treatment with rHuEPO in otherwise untreated rats increased the cHb within one week to 153 g/l (FIG. 1). A subsequent application of carboplatin seven days after commencement of rHuEPO therapy reduced the hemoglobin level within nine days to values comparable to the cHb of untreated control animals. Withdrawal of further rHuEPO application led to the continued development of anemia (FIG. 1). Thus, rHuEPO therapy for approximately one week prior to carboplatin application resulted in prevention (or reversal) of the carboplatin-induced anemia at the time of chemotherapy of the tumor.

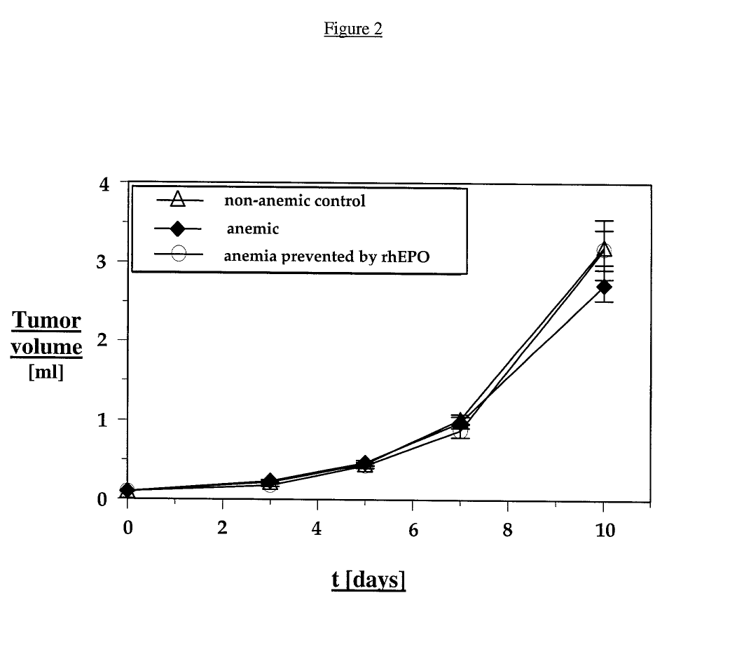

The growth curves of tumors not treated with cyclophosphamide chemotherapy were more or less identical in the non-anemic and anemic group, as well as in the group where anemia was prevented by rHuEPO (FIG. 2). The volume doubling time was approximately 1.8 days independent of the actual cHb or treatment with rHuEPO for sixteen days (FIG. 2). From these results which are in good accordance with data obtained in previous studies [D. K. Kelleher, U. Matthiensen, O. Thews, and P. Vaupel, “Blood Flow, Oxygenation, and Bioenergetic Status of Tumors After Erythropoietin Treatment in Normal and Anemic Rats”, Cancer Res., (1996) 56:4728-4734; O. Thews, R. Koenig, D. K. Kelleher, J. Kutzner, and P. Vaupel, “Enhanced Radiosensitivity in Experimental Tumours Following Erythropoietin Treatment of Chemotherapy-Induced Anaemia”, Br. J. Cancer, (1998) 78:752-756], it can also be concluded that anemia induction by carboplatin (four days prior to tumor implantation), as well as rHuEPO treatment have no impact per se on the growth rate of the tumors under investigation.

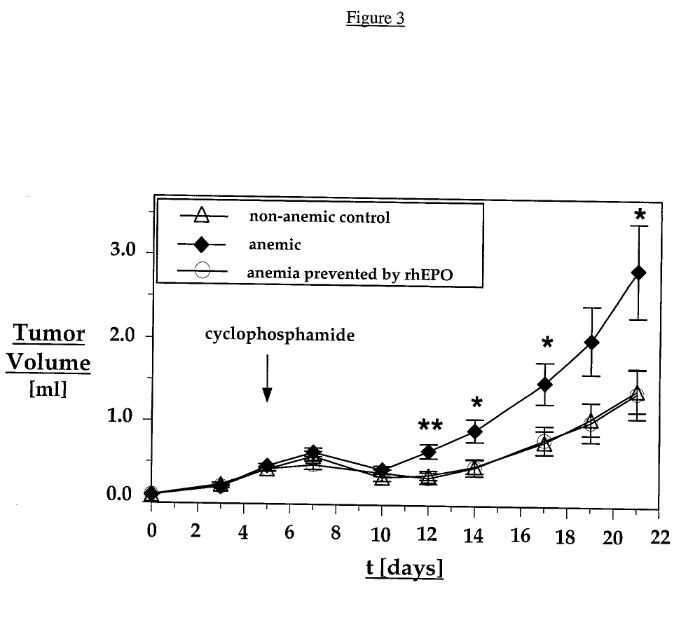

FIG. 3 shows the tumor growth rate in the three groups when tumors were treated with a single dose of cyclophosphamide on Day 5 after tumor implantation. After cyclophosphamide treatment, tumor growth was temporarily arrested and, in some cases, a slight shrinkage of the tumor was observed. After a period of six to nine days, the tumors started to regrow. On the day of cyclophosphamide treatment, RBC-related parameters were significantly different between the two groups where anemia was induced by carboplatin (Table 1), with the anemic group showing a mean cHb of 98 g/l and the group in which anemia development was prevented by rHuEPO administration showing a cHb of 137 g/l (comparable to the cHb in the non-anemic control group with a cHb of 141 g/l). In both anemia groups the parameters describing red blood cells indices (MCV, MCH, MCHC) showed only minor differences and were within the normal range indicating a normocytic, normochromic anemia induced by carboplatin (Table. 1).

TABLE 1
RBC-Related Parameters on the day of Cyclophosphamide treatment
in the anemic (Carboplatin-treated) group, the Carboplatin group where
anemia was prevented by rHuEPO treatment and in non-anemic,
otherwise unheated animals.

| | | CARBOPLATIN | |
|---|---|---|---|
| | CARBOPLATIN- | + rHuEPO | UNTREATED |
| | TREATED | TREATED | CONTROLS |
| N | 12 | 12 | 11 |
| CHb [g/l] | 98 ± 3 | 137 ± 5 | 141 ± 3 |

p < 0.001

| | | | |
|---|---|---|---|
| Hematocrit [%] | 28 ± 1 | 42 ± 2 | 43 ± 1 |

| | p < 0.001 | | |
|---|---|---|---|
| RBC count [10⁶/μl] | 4.5 ± 0.2 | 6.2 ± 0.2 | 6.8 ± 0.1 |
| | p < 0.001 | | |
| Mean Corpuscular Volume | 63 ± 1 | 67 ± 1 | 63 ± 1 |
| (MCV) [fl] | p = 0.002 | | |
| Mean Corpuscular Hemoglobin | 22 ± 1 | 22 ± 1 | 21 ± 1 |
| (MCH) [pg] | n.s. | | |
| Mean Corpuscular Hb | 348 ± 5 | 331 ± 3 | 331 ± 5 |

| | |
|---|---|
| Concentration (MCHC) [g/l] | p = 0.015 |

(n: number of animals; p: values for the comparison of anemic control vs. rHuEPO-group).

FIG. 3 also shows the regrowth characteristics after cyclophosphamide treatment in the anemic group, the group where anemia was reversed by rHuEPO and the non-anemic group. The growth delay following chemotherapy (at the 1.3 ml tumor volume level) was approximately 13.3 days both for the non-anemic and the rHuEPO-treated group, but only 8.6 days in the anemic group. During the regrowth period, the tumor growth rate in the anemic group was slightly higher than in rHuEPO-treated or non-anemic animals (volume doubling time during the regrowth period 4.0 days in the anemic, 4.6 days in the non-anemic group and 4.3 days in rHuEPO-treated animals). All results were confirmed in two independent experimental series.

### Discussion

Since the growth rate of DS-sarcomas in carboplatin-treated control animals (not treated with cyclophosphamide) was almost identical to that of untreated animals, (FIG. 2) it can be concluded that administration of carboplatin at this dose four days before tumor implantation has no effect on the growth rate of DS-sarcomas, which might be the result of the relatively short biological half-life of carboplatin (approximately 3 to 4 hours in humans [P. A. Reece, J. F. Bishop, I. N. Olver, I. Stafford, B. L. Hillcoat, and G. Morstyn, “Pharmacokinetics of Unchanged Carboplatin (CBDCA) in Patients with Small Cell Lung Carcinoma”, Cancer Chemother. Pharmacol., (1987) 19:326-330]). The growth curves of tumors in control animals also show that rHuEPO (epoetin alpha) applied for two weeks at three times per week at a dose of 1000 IU/kg body weight has no impact on the tumor growth per se. These results are in good accordance with previous studies using epoetin beta treatment in the same animal and tumor model.

Anemia with a comparable hemoglobin concentration as in the present study has been shown to result in a worsening of the oxygenation status of experimental tumors. Kelleher et al. [D. K. Kelleher, U. Matthiensen, O. Thews, and P. Vaupel, “Blood Flow, Oxygenation and Bioenergetic Status of Tumors After Erythropoietin Treatment in Normal and Anemic Rats”, Cancer Res., (1996) 56:4728-4734] found, using in the same tumor model, that the fraction of hypoxic pO₂ values between 0 and 2.5 mmHg increased substantially (from 21% to 76%) upon induction of a moderate anemia. These results have recently been confirmed for human squamous cell carcinomas of the head and neck region [A. Becker, P. Stadler, R. S. Lavey, G. Hänsgen, T. Kuhnt, C. Lautenschlager, H. J. Feldmann, M. Molls, J. Dunst, “Severe Anemia is Associated with Poor Tumor Oxygenation in Head and Neck Squamous Cell Carcinomas”, Int. J. Radiat. Oncol. Biol. Phys. (2000) 46:459-466]. Kelleher et al. also demonstrated that correcting anemia by rHuEPO treatment could improve the oxygenation status as indicated by a reduction in the fraction of hypoxic pO₂ values ≦2.5 mmHg to 55% [D. K. Kelleher, U. Matthiensen, O. Thews, and P. Vaupel, “Blood Flow, Oxygenation and Bioenergetic Status of Tumors After Erythropoietin Treatment in Normal and Anemic Rats”, Cancer Res., (1996) 56:4728-4734]. Previous studies have demonstrated that an anemia-related worsening of tumor oxygenation seems to be at least partially responsible for the reduced efficacy of standard radiotherapy [O. Thews, R. Koenig, D. K. Kelleher, J. Kutzner, and P. Vaupel, “Enhanced Radiosensitivity in Experimental Tumours Following Erythropoietin Treatment of Chemotherapy-Induced Anaemia”, Br. J. Cancer, (1998) 78:752-756]. In turn, correction of anemia by rHuEPO was able to improve the radiosensitivity. These results might explain the poorer prognosis of anemic patients following standard radiotherapy [C. Grau and J. Overgaard, “Significance of Hemoglobin Concentration for Treatment Outcome”, In: M. Molls, P. Vaupel (eds.), “Blood Perfusion and Microenvironment of Human Tumors—Implications for Clinical Radiology”, Springer-Verlag, (1998), 101-112, Berlin].

Hypoxia in solid tumors is one major reason for limited efficacy of several non-surgical treatment modalities such as sparsely ionizing radiation [R. S. Bush, R. D. T. Jenkin, W. E. C. Allt, F. A. Beale, A. J. Dembo, and J. F. Pringle, “Definitive Evidence for Hypoxic Cells Influencing Cure in Cancer Therapy”, Br. J. Cancer, (1978) 37(3):302-306] or photodynamic therapy [B. W. Henderson and V. H. Fingar, “Relationship of Tumor Hypoxia and Response to Photodynamic Treatment in an Experimental Mouse Tumor”, Cancer Res., (1987) 47:3110-3114]. In addition, in cell culture experiments, Teicher et al. [B. A. Teicher, J. S. Lazo and A. C. Sartorelli, “Classification of Antineoplastic Agents by Their Selective Toxicities Toward Oxygenated and Hypoxic Tumor Cells”, Cancer Res., (1981) 41:73-81] found that several chemotherapeutic agents were also more effective in the presence of oxygen. These early results have been confirmed by the same group in vivo where the anti-neoplastic activity of several drugs was correlated to the local perfusion in experimental sarcomas of mice [B. A. Teicher, S. A. Holden, A. Al-Achi and T. S. Herman, “Classification of Antineoplastic Treatments by Their Differential Toxicity Toward Putative Oxygenated and Hypoxic Tumor Subpopulations in vivo in FSaIIC Murine Fibrosarcoma”, Cancer Res., (1990) 50:3339-3344]. The authors assumed that well-perfused tissue areas correspond to normoxic regions whereas poorly perfused regions indicate hypoxic tissue. Teicher et al. found that several chemotherapeutic agents (such as cyclophosphamide, carboplatin, melphalan) induced a more pronounced cell kill in “oxygenated” tumor areas than in “hypoxic” regions indicating a possible O₂-sensitivity of these drugs.

Various reasons for these differences has been discussed: (1) oxygen might directly influence the mechanism of action (pharmacodynamics) of anti-neoplastic drugs (e.g., alkylating agents) [B. A. Teicher, “Hypoxia and Drug Resistance”, Cancer Metastasis Rev., (1994) 13:139-168]; (2) hypoxia can cause a cell-cycle arrest and in turn reduce the efficacy of agents acting only on proliferating cells [D. J. Chaplin, M. R. Horsman, M. J. Trotter and D. W. Siemann, “Therapeutic Significance of Microenvironmental Factors”, In: M. Molls and P. Vaupel (eds.), “Blood Perfusion and Microenvironment of Human Tumors”, Springer, (1998) 131-143, Berlin]; (3) hypoxic tissues show a pronounced extracellular acidosis [P. Vaupel, F. Kallinowski and P. Okunieff, “Blood Flow, Oxygen and Nutrient Supply, and Metabolic Microenvironment of Human Tumors: A Review”. Cancer Res., (1989) 49:6449-6465] which might influence the intra-/extracellular drug distribution.

In the present study the sensitivity of experimental tumors to cyclophosphamide was studied. Cyclophosphamide was chosen because Teicher et al. demonstrated a pronounced oxygen sensitivity of this drug and since the DS sarcoma has been shown to be sensitive to treatment with this agent [M. Busse and P. W. Vaupel, “The Role of Tumor Volume in ‘Reoxygenation’ Upon Cyclophosphamide Treatment”, Acta Oncol., (1995) 34:405-408]. The results of the present study clearly indicate that clinically relevant anemia (cHb=98 g/l) results in a worsening of the sensitivity of DS sarcomas to cyclophosphamide treatment. In turn, reversing anemia by rHuEPO treatment significantly increases the cytotoxicity of this chemotherapy (FIG. 3).

Silver and Piver [D. F. Silver and M. S. Piver, “Effects of Recombinant Human Erythropoietin on the Antitumor Effect of Cisplatin in SCID Mice Bearing Human Ovarian Cancer: A Possible Oxygen Effect”, Gynecol. Oncol., (1999) 73:280-284] found in an animal study that the efficacy of cisplatin treatment on xenotransplanted tumors was increased (as indicated by a lower tumor volume) when mice were simultaneously treated with EPO for 3 to 4 weeks. The authors attributed the improved chemosensitivity to a better oxygenation status of the tumor as a result of the higher hemoglobin concentration. However, in their study, animals were not anemic on the days of cisplatin treatment and no oxygen tension measurements have been performed in the tumor to clearly demonstrate an “oxygen effect” of EPO treatment

Since in the present study anemia (which is correlated with a worsening of the oxygenation status) resulted in a reduced efficacy of cyclophosphamide chemotherapy and reversal of anemia by rHuEPO improved the effectiveness of this chemotherapy, the differences in chemosensitivity reported might be at least partially due to an improvement in tumor oxygenation as a consequence of anemia correction. These results form a basis for improving the outcome of chemotherapy in anemic patients by rHuEPO treatment, especially in patients where tumors are known to be hypoxic.

### EXAMPLE 2

Elevation of Hemoglobin Levels in Tumor-Bearing Rats as a Method to Increase Chemotherapeutic/Radiotherapeutic Efficacy

It has been known that hypoxic tissue is refractory to both ionizing radiation and chemotherapy [Brown, (1999) Cancer Research 59:5863-5870]. The mechanism by which this occurs is most likely due to both genetic and physiological parameters. Firstly, hypoxia-induced gene expression as a consequence of tumor progression may up-regulate genes that are negatively correlated with antitumor intervention. Secondly, as the size of the tumor increases resulting in aberrant blood flow, delivery of oxygen and anticancer agents will be impeded. Taken together, systemic administration of erythropoietin to increase the oxygen state of the tumor microenvironment should yield increased anticancer efficacy.

Male Sprague-Dawley rats are injected with a tumor cell line (0.5-1.0×10⁻⁶ cells) in the hind quarter. Concurrent with tumor cell implantation, erythropoietin is administered (1000 IU/kg) three times weekly to increase hemoglobin levels to 2 g/dL above baseline value at study initiation. Once this level is reached, (≈2 weeks after erythropoietin initiation) animals are maintained at elevated hemoglobin levels by weekly administration of erythropoietin 600 IU/kg weekly. Control animals will receive an equivalent volume of saline. All animals will be treated with chemotherapeutic agent two weeks post implantation/erythropoietin initiation and monitored for tumor size every two days for a one month post-study initiation.

What is claimed is:

1. A method to treat a subject in need thereof comprising the steps:
a) administration of a therapeutically effective amount of erythropoietin sufficient to produce at least a physiological hemoglobin level in the subject; and
b) administration of a therapeutically effective amount of cyclophosphamide,
wherein the subject has a solid tumor and where the increased hemoglobin level increases the therapeutic efficacy of the cyclophosphamide.

2. The method of claim 1 wherein the erythropoietin is administered to the subject in the range of about 50 to 1000 IU/kg.

3. The method of claim 1 wherein the erythropoietin is administered to a male subject to produce a hemoglobin level in the subject that is greater than about 15 g/ dL.

4. The method of claim 1 wherein the erythropoietin is administered to a male subject to produce a hemoglobin level in the subject that is greater than about 16 g/ dL.

5. The method of claim 1 wherein the erythropoietin is administered to a male subject to produce a hemoglobin level in the subject that is greater than about 17 g/dL.

6. The method of claim 1 wherein the erythropoietin is administered to a female subject to produce a hemoglobin level in the subject that is greater than about 13 g/dL.

7. The method of claim 1 wherein the erythropoietin is administered to a female subject to produce a hemoglobin level in the subject that is greater than about 14 g/ dL.

8. The method of claim 1 wherein the erythropoietin is administered to a female subject to produce a hemoglobin level in the subject that is greater than about 15 g/ dL.

9. A method to treat a subject in need thereof comprising the steps:
a) administration of a therapeutically effective amount of erythropoietin; and
b) administration of a therapeutically effective amount of cyclophosphamide,
wherein the subject has a solid tumor and where the increased hemoglobin level increases the therapeutic efficacy of the cyclophosphamide.

10. The method of claim 9 wherein the erythropoietin is administered to the subject in the range of about 50 to 1000 IU/kg.

11. The method of claim 9 wherein the erythropoietin is administered to a male subject to produce a hemoglobin level in the subject that is greater than about 15 g/dL.

12. The method of claim 9 wherein the erythropoietin is administered to a male subject to produce a hemoglobin level in the subject that is greater than about 16 g/dL.

13. The method of claim 9 wherein the erythropoietin is administered to a male subject to produce a hemoglobin level in the subject that is greater than about 17 g/dL.

14. The method of claim 9 wherein the erythropoietin is administered to a female subject to produce a hemoglobin level in the subject that is greater than about 13 g/dL.

15. The method of claim 9 wherein the erythropoietin is administered to a female subject to produce a hemoglobin level in the subject that is greater than about 14 g/dL.

16. The method of claim 9 wherein the erythropoietin is administered to a female subject to produce a hemoglobin level in the subject that is greater than about 15 g/dL.
